(19) United States
(12) Patent Application Publication
(10) Pub. No.: US 2008/0214934 A1
(43) Pub. Date: Sep. 4, 2008

(54) Inter-frame processing for contrast agent enhanced medical diagnostic ultrasound imaging

(75) Inventors: Chi-Yin Lee, Bellevue, WA (US); James E. Chomas, San Francisco, CA (US); Ismayil M. Guracar, Redwood City, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/713,209

(22) Filed: Mar. 2, 2007

(51) Int. Cl. A61B 8/00

(52) U.S. Cl. 600/437

(57) ABSTRACT

Contrast agent enhanced medical diagnostic imaging is improved by selecting particular frames of data. Frames of data are acquired over time. Information from the frames of data are combined, such as for a time intensity curve or maximum intensity processing. Rather than combining information from each of the frames, information from some frames is not used. Frames are selected for inclusion. In one embodiment, the selection is based on one type of data (e.g., B-mode) for combining information for another type of data (e.g., contrast agent data).

### BACKGROUND

The present embodiments relate to contrast agent enhanced medical diagnostic ultrasound imaging. In particular, combination of contrast agent image information over time is enhanced.

Imaging blood perfusion in organs or tissue may be useful. In some applications, frames of data acquired over time are integrated. The resulting image may provide useful information for diagnosis, such as showing smaller vessels or perfusion channels.

Some example combinations are maximum intensity holding/processing (MIP), minimum intensity holding, and the construction of a time intensity curve (TIC). U.S. Pat. No. 6,676,606 shows maximum intensity persistence for showing the buildup of micro-bubble tracks through vasculature. A slow decay fades the tracks to black over time. U.S. Pat. No. 6,918,876 teaches intermittent scanning repeated in synchronism with the R-wave. Maximum intensity persistence combines the high luminance contrast portion over time. TIC charts intensity (e.g., B-mode intensity) for a pixel or region of interest as a function of time. The chart shows the in-flow, out-flow, or both of contrast agents over the time associated with the component frames of data. However, due to operator motion or internal motion, the combination of information from different frames may result in blurred images or inaccurate information.

### BRIEF SUMMARY

By way of introduction, the preferred embodiments described below include methods, systems, computer readable media, and instructions for contrast agent enhanced medical diagnostic imaging. Frames of data are acquired over time. Information from the frames of data are combined, such as for TIC or MIP. Rather than combining information from all of the frames, information from some frames is not used. Frames are selected for inclusion, such as based on motion displacement or similarity. In one embodiment, the selection is based on one type of data (e.g., B-mode) for combining information for another type of data (e.g., contrast agent data).

In a first aspect, a method is provided for contrast agent enhanced medical diagnostic ultrasound imaging. A sequence of ultrasound frames of data representing, at least in part, information from contrast agents is generated. A subset of the ultrasound frames of data is selected as a function of a characteristic represented by a first type of data. Information from the selected subset and not from unselected ones of the ultrasound frames of data is combined. The combined information is associated with a second type of data different than the first type of data.

In a second aspect, a computer readable storage medium has stored therein data representing instructions executable by a programmed processor for contrast agent enhanced medical diagnostic ultrasound imaging. The storage medium includes instructions for: selecting frames of ultrasound data associated with less inter frame motion and not selecting frames of data associated with more inter frame motion; integrating the selected frames of ultrasound data as a function of time; and using characteristics of at least a first type of data for the selecting and information of at least a second type of data for the integrating.

In a third aspect, a method is provided for contrast agent enhanced medical diagnostic ultrasound imaging. Frames of data representing a region are acquired over time with ultrasound. The region has some contrast agents. Some of the frames of data are discarded as a function of similarity between the frames of data. An image is formed from the remaining frames of data.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound imaging system for contrast agent enhanced imaging;

FIG. 2 is a flow chart diagram of a method for contrast agent enhanced diagnostic medical ultrasound imaging according to one embodiment;

FIG. 3 is a graphical representation of correlating data without motion compensation in one embodiment;

FIG. 4 is a graphical representation of correlating data with motion compensation in one embodiment

FIG. 5 is a graphical representation of one embodiment of motion displacement;

FIG. 6 is a graphical representation of a displacement curve according to one example;

FIG. 7 is an example reference image;

FIG. 8 is an example MIP of 32 frames of data with no motion correction;

FIG. 9 is an example MIP of the 32 frames of data of FIG. 8 with motion correction; and

FIG. 10 is an example MIP with selection of a subset of frames.

### DETAILED DESCRIPTION OF THE DRAWINGS AND PRESENTLY PREFERRED EMBODIMENTS

Maximum intensity holding for an image sequence is a tool for tracing contrast agents (e.g., micro-bubbles). It is difficult to increase the contrast for perfusion associated with small vessels. Performing MIP on contrast agent images may improve the visibility of the vascular structure. For example, the intensity of each pixel in an MIP image is determined by taking the maximum of the pixel intensity values over time from a plurality of frames. However, due to operator motion and/or internal motion, the MIP may be blurred.

Motion correction between each new frame and a reference frame may reduce the blurring. However, certain forms of motion, such as out-of-plane motion, may not be corrected. Some blurring may still exist. To further reduce blurring or image artifacts, frame selection is performed based on the data acquired. Frames associated with substantial motion are not used in the combination, resulting in less blurring. Frame selection determines whether to integrate the information of a next frame for processing. The frames are selected based on similarity between frames, motion displacement parameters, or other characteristics.

Contrast agent exams in radiology may span many minutes or hundreds of ultrasound frames. There is significant value in reducing the hundreds of frames into one or a plurality of high contrast frames generated in real-time or off-line. In order to maintain high resolution, “bad” frames are thrown out.

In one embodiment, data from one track (e.g. B-mode data) is used to determine motion before performing the MIP process. The MIP process uses at least data acquired in another track (e.g. contrast agent imaging data). Characteristics from one track are used to condition the integration of another track. The tracks correspond to different processing using the same or different hardware path. Using dual tracks of acquired data (e.g., B-mode and contrast agent mode) may produce better inter-frame integration results. Alternatively, the same data or same type of data is used for selecting images and for combination over time.

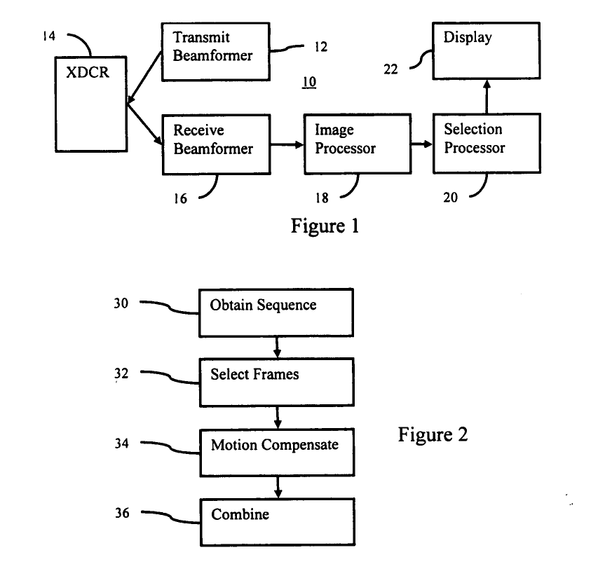

FIG. 1 shows a system 10 for enhanced contrast agent medical diagnostic ultrasound imaging. The system 10 includes a transmit beamformer 12, a transducer 14, a receive beamformer 16, an image processor 18, a selection processor 20, and a display 20. Additional, different, or fewer components may be provided. For example, a separate memory is provided for buffering or storing frames of data over time. As another example, the selection processor 20 is combined with or part of the image processor 18. The system 10 is a medical diagnostic ultrasound imaging system in one embodiment, but other imaging systems of the same (ultrasound) or different modality may be used. In other embodiments, part or all of the system 10 is implemented in a computer or workstation. For example, previously acquired frames of data are processed without the beamformers 12, 16 or transducer 14.

The transmit beamformer 12 is an ultrasound transmitter, memory, pulser, analog circuit, digital circuit, or combinations thereof. The transmit beamformer 12 is operable to generate waveforms for a plurality of channels with different or relative amplitudes, delays, and/or phasing. Upon transmission of acoustic waves from the transducer 14 in response to the generated waves, one or more beams are formed. The transmit beamformer 12 may cause the beam to have a particular phase and/or amplitude. For example, the transmit beamformer 12 transmits a sequence of pulses associated with a given scan line or to adjacent scan lines. The pulses correspond to beams with different amplitudes and/or relative phases. In alternative embodiments, a single beam is used for any given scan line and/or beams with a same amplitude and/or relative phases are used.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. The elements connect with channels of the transmit and receive beamformers 12, 16.

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to each transmission. In alternative embodiments, the receive beamformer 16 is a processor for generating samples using Fourier or other transforms.

The receive beamformer 16 may include a filter, such as a filter for isolating information at a second harmonic or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental or other band.

Any desired sequence of transmit and receive operation may be used to obtain ultrasound information. For example, B-mode data may be obtained by scanning a region once. The B-mode may be used for tissue imaging. Correlation or motion tracking may be used to derive fluid information from B-mode data. B-mode operation may provide contrast agent information. Doppler information may be obtained by transmitting sequences of beams along each scan line. A corner turning memory may be used to isolate tissue, contrast agents, and/or flow information from Doppler signals. Other now known or later developed modes may be used.

In one embodiment, the mode is a contrast agent imaging mode. Contrast agents may be imaged with typical B-mode or Doppler techniques. Isolating information at the second, even, odd, sub, or other harmonics may more likely identify information from contrast agents. For example, a two pulse technique is used. The pulses have a same amplitude, but different phase. By summing the response, information associated with even harmonics is identified. Filtering may alternatively be used. Alternatively or additionally, relative phasing is provided in the receive processing.

In one embodiment, the transmit sequence is controlled to generate echo signals responsive to the cubic fundamental. The beamformer 12 is operable to transmit a plurality of pulses having at least two different amplitude levels and at least two of the plurality of pulses having opposite or different phases. Transmitter power can be varied in any suitable manner, as for example by adjusting the voltage applied to individual transducer elements, or by adjusting the number of transducer elements (or transmit aperture) used to form a particular pulse.

For obtaining ultrasound data at the cubic fundamental, the receive beamformer 16 includes line memories and a summer or a filter to combine signals responsive to the transmissions. The line memories or buffers can be formed as physically separate memories, or alternately they can be formed as selected locations in a common physical device. The beamformed signals are stored in the line memories or buffers and then weighted and summed in a weighted summer. Weighting values for both amplitude and phase are used in the weighted summer. The memories and the summer can be implemented using analog or digital techniques. The weighted summer forms a composite output signal by weighting the separate beamformed receive signals. The composite output signal for a given spatial location is a sample associated with the cubic fundamental response.

Obtaining cubic fundamental information is disclosed in U.S. Pat. No. 6,494,841, the disclosure of which is incorporated herein by reference. Any of the transmit sequences and receive combinations disclosed therein may be used for obtaining cubic fundamental information. Other transmit sequences and receive combinations for obtaining cubic fundamental information may be used, such as disclosed in U.S. Pat. Nos. 6,602,195, 6,632,177, 6,638,228 and 6,682,482, the disclosures of which are incorporated herein by reference. In general, a sequence of pulses with different amplitudes and phases are transmitted. Using amplitude change or different amplitudes without different phases may also be used to obtain cubic fundamental information. By combining received signals responsive to the sequence, a sample including cubic fundamental information is obtained. The cubic fundamental information is highly specific to ultrasound contrast agents since contrast agents produce cubic response and the transducer and tissue produce very little cubic response. The information provides tissue clutter rejection, allowing for imaging more specific to contrast agents. For example, small vessels within tissue may be more easily imaged or identified using cubic fundamental information.

The image processor 18 is a B-mode detector, Doppler detector, pulsed wave Doppler detector, correlation processor, Fourier transform processor, application specific integrated circuit, general processor, control processor, field programmable gate array, digital signal processor, analog circuit, digital circuit, combinations thereof or other now known or later developed device for detecting information for display from beamformed ultrasound samples.

In one embodiment, the image processor 18 implements a fast Fourier transform from a plurality of samples representing a same region or gate location. Each of the samples is responsive to cubic fundamental so that a pulsed wave Doppler display may be generated from cubic fundamental information. The image processor 18 also includes a B-mode detector in a parallel track. The B-mode detector operates on the same or different beamformed samples to detect tissue, contrast agent, or tissue and contrast agent response. For example, one receive beam for each spatial location from the sequence of receive beams used for cubic fundamental isolation is applied to the B-mode detector for imaging primarily tissue information.

The image processor 18 outputs frames of ultrasound data. The frames of data are formatted in an acquisition format (e.g., polar coordinate), a display format (e.g., scan converted into a Cartesian coordinate format or an image), or other format. Each frame of data represents a one, two, or three-dimensional scanned region. The frames of data include a single or multiple types of data. For example, one frame of data includes just contrast agent information. As another example, one frame of data includes contrast agent information for some spatial locations and another type of information (e.g., B-mode or Doppler) for other spatial locations. Different types of data may be provided in the same frame for a same spatial location. In another example, the different types of data are provided in different frames of data.

In an alternative embodiment, the image processor 18 loads data from a network or memory. For example, DICOM or other images are loaded. Each image is a frame of data. One frame may include different types of data, one overlaid on another. Alternatively, each frame includes only one type of data with different frames for different data types. In another embodiment, each frame is subdivided so that one portion includes one type of data and another portion includes another type of data.

The selection processor 20 is an application specific integrated circuit, correlation processor, Fourier transform processor, general processor, control processor, field programmable gate array, digital signal processor, analog circuit, digital circuit, combinations thereof, or other now known or later developed device for determining similarity and/or displacement between frames of data. The selection processor 20 receives the frames of data to determine which frames should be included in MIP, TIC, or other images generated from combinations of information from frames of data.

The selection processor 20 may also include a persistence filter, other filter, summer, alpha blending buffer, other buffer, memory, processor, adder, or other device for generating an image from information of different frames of data. For example, the selection processor 20 compares data for a particular spatial location from one frame to another frame or an ongoing combination frame. Based on the comparison (e.g., highest value, contribution to mean value, or lowest value), one of the values is selected or the ongoing combination frame is updated to include the desired value. As another example, the selection processor 20 determines an average, total, or other value representing a location or region as a function of time.

The display 20 is a CRT, monitor, LCD, flat panel, projector or other display device. The display 20 receives display values for displaying an image. The display values are formatted as a one-dimensional image, two-dimensional image, or three-dimensional representation. In one embodiment, the display values are for an image generated as a function of frames of data acquired at different times, such as a TIC or MIP image. As additional frames of data are acquired and selected, the image may be updated. Other images, such as images from single or component frames of data, may also be displayed.

The image processor 18 and/or selection processor 20 operate pursuant to instructions. A computer readable storage medium stores data representing instructions executable by one or both of these programmed processors for contrast agent enhanced medical diagnostic ultrasound imaging. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

FIG. 2 shows a method for contrast agent enhanced medical diagnostic ultrasound imaging. The method is implemented by the system 10 of FIG. 1 or a different system. The method is performed in the order shown or a different order. Additional, different, or fewer acts may be provided, such as not providing act 34 and/or 36.

In act 30, a sequence of ultrasound frames of data is generated. The sequence is generated by acquiring frames of data with ultrasound, or by acquiring previously generated frames of data (e.g., DICOM images). The frames of data are acquired in real time with live scanning or are from stored clips. The sequence may be substantially continuous or periodic (e.g., acquired once or more every heart cycle).

The sequence includes frames of data representing a scanned region at different times. Each frame of data represents a same or overlapping region. Some frames may represent different regions, such as due to out-of-plane motion of the transducer relative to the patient.

The region includes contrast agents or an area likely to include contrast agents after insertion of the agents. The contrast agents respond to ultrasound energies. Some or all of the frames of data include information from contrast agents. The information may also include response from tissue or fluids. In one embodiment, the information is obtained at a cubic fundamental of ultrasound signals. For example, ultrasound signals are transmitted in a plurality of pulses having at least two different amplitude levels and phases. To avoid or minimize destruction of the contrast agents, low amplitude transmissions (e.g., MI less than 0.7) are used. Signals responsive to the transmissions are combined. Data is acquired at each spatial location of a region of interest in each frame of data.

Only one type of data is represented in the frames of data, such as data representing just contrast agents or responses from contrast agent and tissue. Alternatively, the frames of data represent different types of data, such as in a same frame or in different sets of frames.

In act 32, a subset of the ultrasound frames of data is selected as a function of a characteristic. Generally, the frames of data associated with less inter frame motion are selected, and frames of data associated with more inter frame motion are not selected. The frames of data with undesired motion are discarded. Any desired threshold may be used. Other criteria may be used.

Motion compensation of act 34 may be applied to the frames of data to correct for in-plane motion between frames. Motion is corrected by determining a relative translation and/or rotation along one or more dimensions. Data from one frame of data is correlated with different regions in the other frame of data to identify a best or sufficient match. The displacement of the data between frames is then used to align the spatial locations between frames. The motion correction may remove or lessen motion associated with transducer movement, patient movement, or organ movement. Global or local motion may be corrected. Alternatively, no motion correction between frames is used.

With or without motion correction of act 34, any one or more characteristic may be used for selecting frames of data in act 32. Frames that undergo smooth motion with respect to the preceding or subsequent frames are picked for combination of information (e.g., the MIP process). Any frame, which has an abrupt motion with respect to another frame, may be excluded.

In one embodiment, a similarity between different frames of data is compared to a threshold. The similarity is between temporally adjacent frames of data. For example, each new frame of data is compared to the immediately preceding, selected frame of data. Alternatively, non-adjacent frames of data are compared.

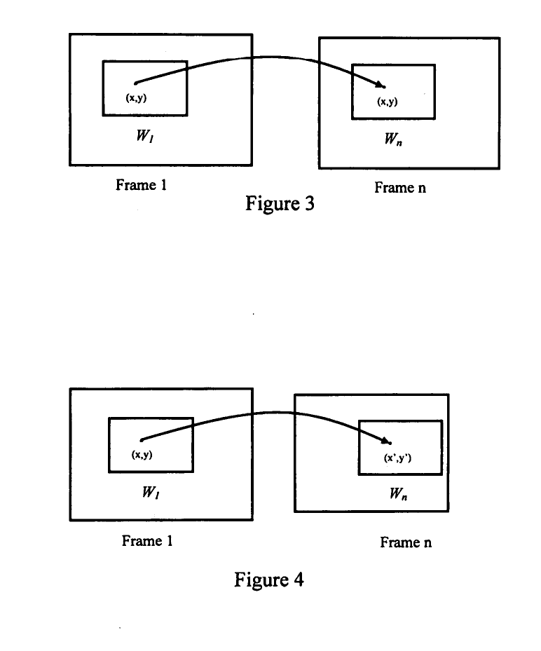

FIG. 3 shows an example embodiment for determining a similarity where motion correction is not used. A matching window, w₀, is specified in a reference frame 1. The reference frame 1 is a selected or desired frame of data. The matching window is the entire frame, a continuous region of the frame, discontinuous region of the frame, multiple regions, or other grouping of spatial locations. In one embodiment, a single window of 100×100 or 150×150 pixels or spatial locations is used, but other sizes may be used. The region may correspond to, cover, or overlap with a region of interest, such as a center of the scanned region. For any newly arrived frame (e.g., Frame n), a matching window, wₙ, at the same location as in the reference Frame 1 is chosen.

FIG. 4 shows an example embodiment for determining the similarity where motion correction is used. A matching window, w₁, is specified on the reference frame 1. For any newly arrived frame n, matching with the reference frame is performed. The motion related displacement determines the placement of the corresponding matching window, wₙ, at the current frame n.

For each new frame of data, the previous or temporally adjacent, selected frame of data is used as the reference frame 1. Alternatively, the same reference frame is used for comparison to each subsequent, even temporally spaced, frames of data.

After the window location is determined, the similarity between the data in the windows is computed. Any similarity function may be used, such as a correlation, cross-correlation, minimum sum of absolute differences, or other function. The similarity is for data within wₙ in the current frame and w₀ in the reference frame. With motion correction, the similarity may be a value associated with the best match.

The frame being compared (i.e., the non-reference frame) is selected or not selected for inclusion as a function of the similarity. If the similarity is higher (e.g., correlation) or lower (e.g., minimum sum of absolute differences) than a threshold, this frame is selected for inclusion. Otherwise, the frame is selected for exclusion or is discarded from the combination processing.

The threshold is predetermined, defined by the user, or adaptive. Predetermined thresholds may be based on experimentation for different imaging applications. User definition allows adjustment of the threshold to provide an image desired by the user. Any adaptive process may be used. For example, contrast agents are allowed to perfuse a region. The user or system then causes destruction by transmitting a higher power beam or beams. The first two frames acquired after destruction are likely similar. This similarity measure with or without an offset (e.g., multiply by 2, 10 or other value or add a value) is used as the threshold for subsequent selection. As another example, a variance between aligned frames of data is used to determine the threshold. Any adaptive threshold is maintained the same for an entire sequence or may adapt throughout the processing of a sequence of frames.

In another embodiment, the frames are selected or not based on a motion displacement between the different frames of data, such as temporally adjacent frames of data. Any now known or later developed technique for determining relative motion between frames of data may be used. For example, a motion sensor on the transducer determines displacement. As another example, a motion correction or compensation technique is used. In another example, a plurality of local motions are combined to determine a global motion.

The motion displacement is along one or more dimensions. Translation and/or rotational displacement may be determined. For example, translation in two dimensions within the imaging plane is determined with or without in-plane rotation.

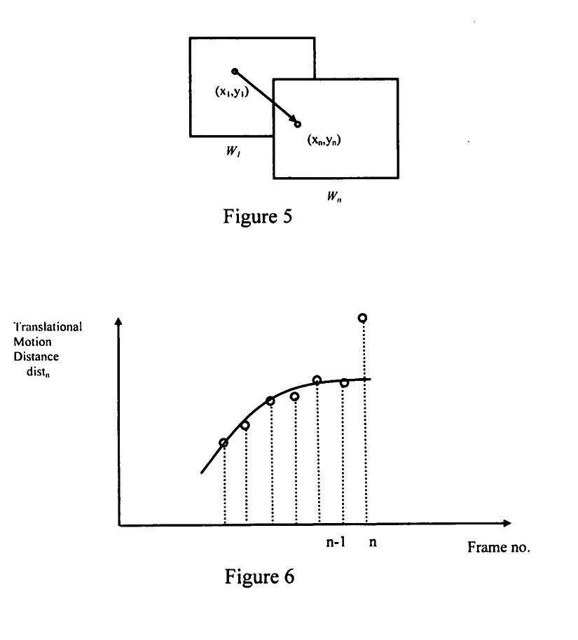

FIG. 5 shows one example of motion displacement. A matching window, w₁, is specified on the reference frame. For any newly arrived frame, motion correction with the reference frame is performed, and the corresponding matching window, wₙ, at the current frame is determined. Similarities at different window positions are determined The arrow represents the translation in-plane between the frames for a best or sufficient match. Given the motion parameters, the translational motion distance motion between w₁ and wₙ is determined. For example, translation motion is determines as follows:

distₙ=\sqrt{(xₙ−x₁)²+(yₙ−y₁)²}

Other calculations may be used.

The amount of displacement between the reference frame and the other frame is used to select or not select the other frame for inclusion. Displacement between temporally adjacent frames or between spaced apart frames is used. The reference frame is the same for all or a plurality displacement calculations or the reference frame is changed, such as associated with a temporally moving window. Differences in or a sum of displacement between different pairs of frames may be used to determine the desired displacement.

A threshold amount of displacement results in inclusion or exclusion. In another embodiment, the displacement relative to other displacements associated with the sequence is provided. For example, the threshold adapts based on displacements. FIG. 6 shows an example of an adaptive displacement threshold. A curve showing the translational motion distance for each frame and the reference frame is plotted. FIG. 6 shows seven displacements by distance as a function of frame or time. For example, the motion correction for Frameₙ has translational motion distance with respect to the reference frame of distₙ. Given the calculated distance values for preceding frames (i.e. dist₁, dist₂, . . . , distₙ₋₁), a curve is fit to the distances. For example, a second degree polynomial or other type of curve is fit. The distance between the current distance (e.g., coordinate (n, distₙ)) and the fit curve is determined. If the distance is smaller than a threshold, the frame is selected. Otherwise, the frame is excluded from the combination process.

In one embodiment, the characteristic for selection relates to or is derived from the data to be combined. In another embodiment, characteristics of at least a first type of data are used for the selecting, and data of at least a second type of data is combined. For example, several clinical ultrasound images or frames of data with mixed contrast agent type data and B-mode type data are used—the B-mode or more tissue responsive data used for selection and the contrast agent or more contrast agent responsive data combined. The different types of data represent the same or overlapping regions at a same or substantially same time. A given type of data may be used for both selecting and combining, such as including the first type of data used for selecting also in the combining. One or both types of data may be exclusive to the combining, selecting or both. A given type of data may be responsive to the same or different types of tissue than another type of data.

In act 34, motion between the frames of data is corrected. The motion compensation or correction is performed before or after selection. For example, the same similarity or displacement calculation is used for selection and motion correction. After determining displacement based on similarity or other information, the frames of data are spatially aligned. Rigid or non-rigid correction may be used. The alignment more likely avoids blurring.

In act 36, information from the selected subset of frames and not from unselected ones of the ultrasound frames of data is combined. The combination is for any now known or later developed inter-frame processing, such as maximum intensity holding, minimum intensity holding, mean determination, or constructing one or more time intensity curves. A new frame of data or image is generated as a function of data from the selected frames. The selected frames of ultrasound data are integrated as a function of time. Integrated includes mathematical integration or forming an image from a plurality of sources.

For each spatial location of a region of interest, the data is compared or used to determine a value. For each pixel of the image, a value is selected as a function of data from each of the remaining (selected) frames of data. For example, the mean, median or other statistical value of data for each spatial location as a function of time is determined from the frames. As another example, the maximum, minimum, or other data in relation to data of the selected frames is selected based on comparison. The frames of the selected subset are combined into a persisted frame or single frame. In another example, a curve representing intensity or other contrast agent response as a function of time is determined from the frames. The curve is for a region or for a spatial location. Since the frames are associated with different times, the curve is of intensity as a function of time.

As new frames are selected, a new persisted or other frame or image is calculated. Alternatively, a single frame is determined for the entire sequence.

The data combined is of the same or different type of data used for selection. For example, contrast agent specific or related data is integrated. A different type of data, such as B-mode data with or without the contrast agent specific data is used for selection.

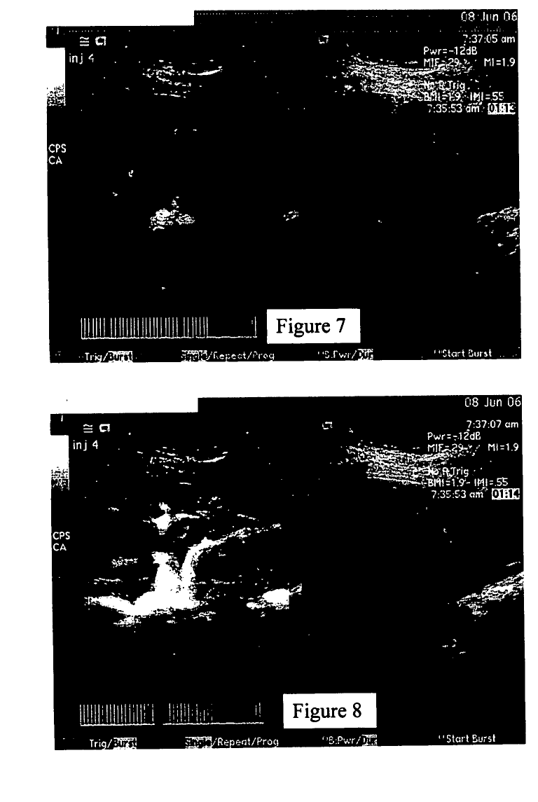

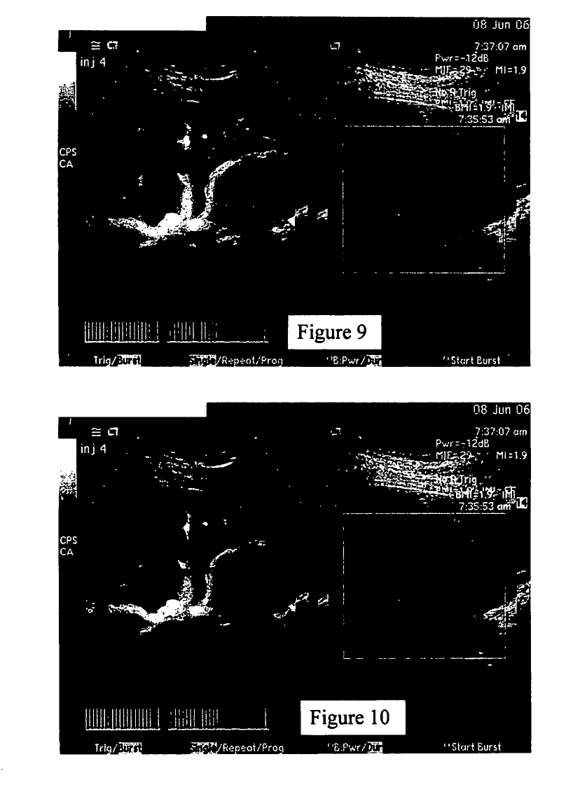

By combining information from contrast agents, such as information primarily at a cubic fundamental of ultrasound signals, the perfusion of contrast agents and/or small vasculature may more easily be viewed. For example, FIGS. 7-10 show maximum intensity processing or combination. In FIG. 7, a reference image is shown with contrast agent information on the left and B-mode information on the right. FIG. 8 shows a combination of contrast agent information for 32 frames of data. The combination is on the left. Motion correction is not used, so blurring occurs. FIG. 9 shows combination of the same contrast agent information for 32 frames of data, but with motion correction. The combination is on the left, and has less blurring than in FIG. 8. FIG. 10 shows combination of 32 selected frames after discarding undesired frames. The combination is on the left, and shows less blurring than in FIG. 9.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for contrast agent enhanced medical diagnostic ultrasound imaging, the method comprising:
generating a sequence of ultrasound frames of data representing, at least in part, information from contrast agents;
selecting a subset of the ultrasound frames of data as a function of a characteristic represented by a first type of data; and
combining information from the selected subset and not from unselected ones of the ultrasound frames of data, the information associated with a second type of data different than the first type of data.

2. The method of claim 1 wherein generating comprises generating the ultrasound frames of data as DICOM images.

3. The method of claim 1 wherein the first type of data is from different ones or different portions of the DICOM images than the second type of data.

4. The method of claim 1 wherein generating comprises obtaining the data as information at a cubic fundamental of ultrasound signals.

5. The method of claim 4 wherein obtaining comprises transmitting the ultrasound signals in a plurality of pulses having at least two different amplitude levels and phases, and combining signals responsive to the transmitting.

6. The method of claim 1 wherein selecting comprises selecting as a function of the characteristic of B-mode data, and combining comprises combining the information from contrast agents.

7. The method of claim 1 wherein selecting comprises:
determining a similarity between different frames of data; and
selecting frames for inclusion as a function of the similarity.

8. The method of claim 1 wherein selecting comprises:
determining a motion displacement between different frames of data; and
selecting frames for inclusion as a function of the motion displacement.

9. The method of claim 1 wherein combining information comprises combining the frames of the selected subset into a persisted frame.

10. The method of claim 1 wherein combining information comprises generating a time intensity curve as a function of time.

11. The method of claim 1 further comprising:
correcting for motion between the frames of data.

12. The method of claim 1 wherein selecting comprises selecting the frames of data associated with less inter frame motion and not selecting frames of data associated with more inter frame motion.

13. In a computer readable storage medium having stored therein data representing instructions executable by a programmed processor for contrast agent enhanced medical diagnostic ultrasound imaging, the storage medium comprising instructions for:
selecting frames of ultrasound data associated with less inter frame motion and not selecting frames of data associated with more inter frame motion;
integrating the selected frames of ultrasound data as a function of time; and
using characteristics of at least a first type of data for the selecting and information of at least a second type of data for the integrating.

14. The instructions of claim 13 wherein using comprises using information primarily at a cubic fundamental of ultrasound signals as the second type of data and B-mode data as the first type of data.

15. The instructions of claim 13 wherein selecting comprises:
determining a similarity between different frames of data; and
selecting frames for inclusion as a function of the similarity.

16. The instructions of claim 13 wherein selecting comprises:
determining a motion displacement between different frames of data; and
selecting frames for inclusion as a function of the motion displacement.

17. The instructions of claim 13 wherein integrating comprises combining the selected frames into a single frame.

18. A method for contrast agent enhanced medical diagnostic ultrasound imaging, the method comprising:
acquiring frames of data representing a region over time, the region having some contrast agents, with ultrasound;
discarding some of the frames of data as a function of similarity between the frames of data; and
forming an image from the remaining frames of data.

19. The method of claim 18 wherein acquiring comprises, for each spatial location represented in each frame of data, transmitting a plurality of pulses having at least two different amplitude levels and phases, and combining signals responsive to the transmitting.

20. The method of claim 18 wherein discarding comprises:
determining a similarity between different, temporally adjacent, frames of data; and
selecting frames for exclusion from the forming as a function of the similarity.

21. The method of claim 18 wherein discarding comprises:
determining a motion displacement between different, temporally adjacent, frames of data; and
selecting frames for exclusion as a function of the motion displacement.

22. The method of claim 18 wherein forming comprises, for each pixel of the image, selecting a value as a function of data from each of the remaining frames of data.

23. The method of claim 18 wherein acquiring comprises acquiring in real-time with ultrasound scanning.
